(19) United States
(12) Patent Application Publication
(10) Pub. No.: US 2025/0281130 A1
(43) Pub. Date: Sep. 11, 2025

(54) PROGRAM AND SYSTEM FOR PROCESSING DETECTION SIGNAL OF RADIATION TRANSMITTED THROUGH IMAGING SUBJECT TO GENERATE RADIOLOGICAL IMAGE

(71) Applicant: GE Precision Healthcare LLC, Waukesha, WI (US)

(72) Inventors: Yasuhiro Imai, Hino (JP); Koichi Hoshino, Hino (JP)

(21) Appl. No.: 19/074,097

(22) Filed: Mar. 7, 2025

(30) Foreign Application Priority Data
Mar. 7, 2024 (JP) 2024-035145

(51) Int. Cl. A61B 6/40; A61B 6/00; A61B 6/03; A61B 6/58

(52) U.S. Cl. CPC A61B 6/405; A61B 6/035; A61B 6/542; A61B 6/583

(57) ABSTRACT

A system, which includes a processor for processing a detection signal of radiation transmitted through an imaging subject to generate a radiological image, is described. The processor receives input for rotation speed and/or number of views per rotation, identifies a tube voltage waveform corresponding to the rotation speed and/or the number of views per rotation using a waveform identification model, calculates a low-energy average of radiation corresponding to a low voltage interval including the low steady-state interval of the tube voltage waveform and a part of the falling part of the tube voltage waveform, and a high-energy average of radiation corresponding to a high voltage interval including the high steady-state interval of the tube voltage waveform and another part of the falling part of the tube voltage waveform, and sets a parameter used for creating the radiological image in accordance with the low-energy average and the high-energy average.

### CROSS-REFERENCE TO RELATED APPLICATIONS

This application claims priority to Japanese Application No. 2024-035145, filed on Mar. 7, 2024, the disclosure of which is incorporated herein by reference in its entirety.

### TECHNICAL FIELD

The present invention relates to a radiation imaging device, and more particularly to a technology for realizing multi-energy imaging.

### BACKGROUND

As an imaging method using a radiation imaging device represented by an X-ray CT device (X-ray Computed Tomography System), there is known an imaging method called a dual-energy imaging method in which data is acquired by switching the radiation tube voltage.

In the case of an X-ray CT device, this imaging method utilizes the concept that the absorption spectrum of X-ray energy differs depending on the substance, in order to obtain images in which specific substances in the subject are emphasized or suppressed. Specifically, for example, a subject including a first substance and a second substance is irradiated with first X-rays and second X-rays having different energy spectra, and first X-ray projection data and second X-ray projection data corresponding to a plurality of views are acquired. Furthermore, a first density image is reconstructed on the basis of the first X-ray projection data, and a second density image is reconstructed on the basis of the second X-ray projection data. Moreover, on the basis of the first density image and the second density image, a dual-energy image is obtained, which is a density distribution image indicating the density distribution of a certain substance, or is a monochromatic image that is an image of a certain energy spectrum.

For example, a method for acquiring projection data while alternately switching the X-ray tube voltage between a low tube voltage and a high tube voltage for each view, in other words, each time the gantry of the X-ray CT device rotates for one view, can be considered as a method for acquiring the first X-ray projection data and the second X-ray projection data as described above.

### SUMMARY

As described above, when acquiring projection data while alternately switching the X-ray tube voltage between a low tube voltage and a high tube voltage, it is desirable to perform instantaneous switching from the low tube voltage to the high tube voltage and from the high tube voltage to the low tube voltage. However, due to limitations in the performance of transformers, a rise time from the low tube voltage to the high tube voltage and a fall time from the high tube voltage to the low tube voltage occur. The rise time and fall time of such a tube voltage waveform are affected not only by the potential difference between the high tube voltage and the low tube voltage, but also by the switching speed between the high tube voltage and the low tube voltage, the magnitude of the tube current, and the like.

Regarding the tube current, whether the tube current is large or small, the rise time does not vary significantly, whereas the fall time is affected more significantly than the rise time. For example, when the tube current is 400 mA, switching from the high tube voltage to the low tube voltage occurs in a relatively short time, but when the tube current is 200 mA, switching from the high tube voltage to the low tube voltage takes a relatively long time. In other words, when the tube current is 400 mA, the fall time is relatively short, and when the tube current is 200 mA, the fall time is relatively short. Furthermore, the rise time and fall time are often curved rather than linear, and when data is sampled continuously, in other words, when a curved section of the tube voltage waveform is included in the sampling, a change occurs in the energy spectrum of an irradiated X-ray beam, and the average energy of the sampled X-ray beam changes.

Furthermore, when the number of views to be acquired is constant, a change in the rotation speed changes the sampling rate, which changes the cycle of switching from the high tube voltage to the low tube voltage. As a result, the duration of the high-voltage steady-state interval of the tube voltage waveform corresponding to the high tube voltage and the duration of the low-voltage steady-state interval of the tube voltage waveform corresponding to the low tube voltage vary. In other words, as the rotation speed increases, the duration of both the high steady-state interval and the low steady-state interval becomes shorter, and as the rotation speed decreases, the duration of both the high steady-state interval and the low steady-state interval becomes longer.

On the other hand, in order to solve the problems of variation in the sensitivity of individual detector elements and deviation of the actual X-ray energy spectrum from the ideal energy spectrum, calibration is performed in accordance with an image reconstruction protocol (preset). As described above, when the rotation speed or the magnitude of the tube current changes, the energy spectrum of the irradiated X-rays varies, and thus it becomes necessary to execute calibration for each type of rotation speed used and each type of tube current used. This requirement leads to a longer downtime when the X-ray CT device cannot be used, a longer imaging time for complicated calibration, a longer calculation time therefor, and increased service costs.

Therefore, there is a demand for a new technique capable of shortening calibration imaging and calculation time while maintaining the image quality of a reconstructed image, reducing the downtime of a radiation imaging device, and reducing service costs.

In the present disclosure, one or more of the problems identified above or related problems are addressed, at least in part, by a system that includes a processor for processing a detection signal of radiation transmitted through an imaging subject to generate a radiological image. Herein, the radiation is irradiated toward the imaging subject while a radiation tube rotates around the imaging subject, and the radiation includes low-energy radiation generated by applying a low tube voltage to the radiation tube, and high-energy radiation generated by applying a high tube voltage to the radiation tube. Application of the low tube voltage and the high tube voltage to the radiation tube are alternately switched during rotation of the radiation tube, to form a tube voltage waveform having a rising part from the low tube voltage to the high tube voltage, a falling part from the high tube voltage to the low tube voltage, a high steady-state interval between the rising part and the falling part, and a low steady-state interval between the falling part and the rising part. A processor receives input for rotation speed and/or number of views per rotation, identifies a tube voltage waveform corresponding to the rotation speed and/or the number of views per rotation using a waveform identification model, calculates a low-energy average value of radiation corresponding to a low voltage interval including the low steady-state interval of the tube voltage waveform and a part of the falling part of the tube voltage waveform, and a high-energy average value of radiation corresponding to a high voltage interval including the high steady-state interval of the tube voltage waveform and another part of the falling part of the tube voltage waveform, and sets a parameter used for creating the radiological image in accordance with the low-energy average value and the high-energy average value.

In another aspect of the present disclosure, one or more of the problems identified above or related problems are addressed, at least in part, by a program for processing a detection signal of radiation transmitted through an imaging subject to generate a radiological image. Herein, the radiation is irradiated toward the imaging subject while a radiation tube rotates around the imaging subject. The radiation includes low-energy radiation generated by applying a low tube voltage to the radiation tube and high-energy radiation generated by applying a high tube voltage to the radiation tube. Application of the low tube voltage and the high tube voltage to the radiation tube are alternately switched during rotation of the radiation tube, to form a tube voltage waveform having a rising part from the low tube voltage to the high tube voltage, a falling part from the high tube voltage to the low tube voltage, a high steady-state interval between the rising part and the falling part, and a low steady-state interval between the falling part and the rising part. A program further causes the processor to execute the following: receiving input for rotation speed and/or number of views per rotation; identifying a tube voltage waveform corresponding to the rotation speed and/or the number of views per rotation using a waveform identification model; calculating a low-energy average value of radiation corresponding to a low voltage interval including the low steady-state interval of the tube voltage waveform and a part of the falling part of the tube voltage waveform, and a high-energy average value of radiation corresponding to a high voltage interval including the high steady-state interval of the tube voltage waveform and another part of the falling part of the tube voltage waveform; and setting a parameter used for creating the radiological image in accordance with the low-energy average value and the high-energy average value.

### BRIEF DESCRIPTION OF THE DRAWINGS

FIG. 1 is a diagram schematically depicting a configuration of an X-ray CT system according to the present embodiment;

FIG. 2 is a diagram depicting a configuration of a main part of an X-ray tube and X-ray detecting part;

FIG. 3 is a diagram depicting the energy distribution of an X-ray beam that changes depending on the tube voltage;

FIG. 4 is a graph depicting a time change in a tube voltage and a graph depicting a time change in a tube current;

FIG. 5 is a diagram depicting a tube voltage waveform;

FIG. 6 is a diagram depicting a tube voltage waveform; and

FIG. 7 is a diagram describing a technique for obtaining a beam hardening correction coefficient to be applied.

### DETAILED DESCRIPTION OF THE DRAWINGS

Embodiments of the present invention will be described below. Note that the invention is not limited thereto.

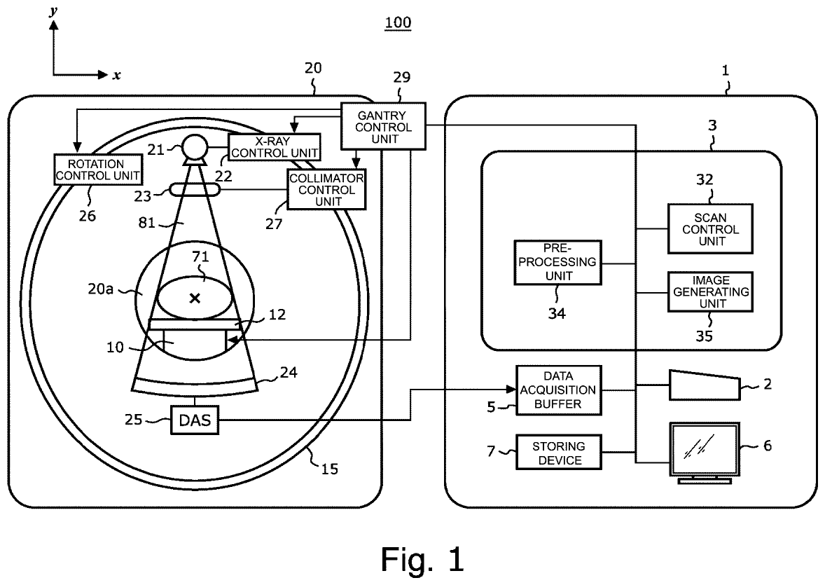

FIG. 1 is a block diagram depicting a configuration of an X-ray CT device 100 according to the present embodiment. In the present disclosure, a medical X-ray CT device is described as an example, but the present invention can be applied to non-destructive examining devices, such as dental CT devices, CT devices for inspecting baggage, and the like. The X-ray CT device 100 includes an operation console 1, an imaging table 10, and a scanning gantry 20.

The operation console 1 has a configuration serving as a computer. Specifically, the operation console 1 includes an input device 2, such as a keyboard, a mouse, or the like for receiving input from an operator; a central processing device 3 for executing scan control processing, pre-processing, image generation processing, and the like; and a data acquisition buffer 5 for acquiring X-ray detector data acquired by the scanning gantry 20. Furthermore, the operation console 1 includes a monitor 6 for displaying a multi-energy image generated by the image generation processing; and a storing device 7 for storing a program, X-ray detector data, X-ray projection data, a dual-energy image, or the like. The imaging conditions are input from the input device 2 and stored in the storing device 7.

The imaging table 10 includes a cradle 12 on which a subject 71 is placed and which is moved in and out of an opening 20a (to be described later) of the scanning gantry 20. The cradle 12 is moved up and down and horizontally in a straight line by a motor internally provided in the imaging table 10.

The scanning gantry 20 has the opening 20a through which the subject 71 to be imaged is transported. Furthermore, the scanning gantry 20 has an X-ray tube 21; an X-ray control unit 22 for controlling the X-ray tube voltage, X-ray irradiation timing, and the like in the X-ray tube 21; and a collimator 23 having an opening that shapes X-rays irradiated from the X-ray tube 21 into a fan-shaped X-ray beam 81. Furthermore, the scanning gantry 20 has a collimator control unit 27 for controlling the opening of the collimator 23; an X-ray detector 24 for detecting X-rays irradiated from the X-ray tube 21; and a data acquisition system (DAS) 25 for acquiring X-ray detector data (also referred to as raw data) from an output of the X-ray detector 24. The DAS 25 samples analog data received from a detector element of the X-ray detector 24 and converts the analog data to digital signals for subsequent processing.

Furthermore, the scanning gantry 20 has a gantry rotating part 15 that holds the X-ray tube 21, the collimator 23, and the X-ray detector 24 and rotates around the body axis of the subject 71; and a rotation control unit 26 that controls the gantry rotating unit 15. Furthermore, the scanning gantry 20 has a gantry control unit 29 for transmitting and receiving control signals between the operation console 1 and the X-ray control unit 22, rotation control unit 26, imaging table 10, and the like. Note that in implementation, the scanning gantry 20 includes: a beam-forming X-ray filter that spatially controls the dose of the X-ray beam 81; and an X-ray filter that controls the radiation quality of the X-ray beam 81. The gantry rotating part 15 holds these filters between the collimator 23 and the opening 20a, and illustrations and detailed descriptions thereof are omitted herein.

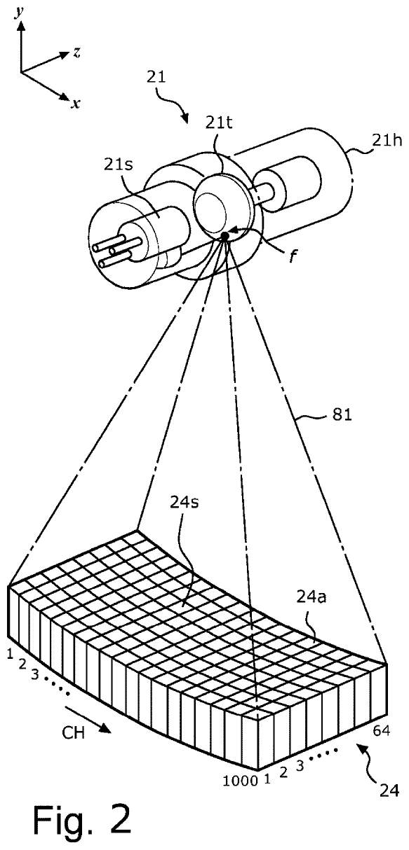

FIG. 2 is a diagram depicting a configuration of a main part of the X-ray tube 21 and X-ray detecting part 24. Herein, the vertical direction is defined as the y-axis direction, a transporting direction of the imaging table 10 (which usually coincides with a thickness direction of the X-ray beam 81 or the body axis direction of the subject 71) is defined as the z-axis direction, and the direction orthogonal to the y-axis and z-axis directions (channel direction) is defined as the x-axis direction.

These components are supported on a prescribed base part of the gantry rotating part 15 and maintain the positional relationship as depicted in the drawings. In other words, the X-ray tube 21 and the X-ray detector 24 are disposed opposite each other with the opening 20a therebetween. Furthermore, X-rays emitted from the X-ray tube 21 pass through a slit formed by the collimator 23 (not depicted in FIG. 2), forming a fan-shaped X-ray beam 81 having a prescribed thickness (cone angle) and spread (fan angle).

The X-ray tube 21 has a structure in which a cathode sleeve 21s incorporating a focusing electrode and a cathode filament, and a rotating target electrode 21t are housed in a housing 21h, and generates X-rays emitted from an X-ray focal point F.

The X-ray detecting part 24 is a so-called multi-row X-ray detector in which a plurality of detecting element rows, for example, 64 detecting element rows, are arranged in the z-axis direction (thickness direction of X-ray beam 81), each detecting element row being obtained by arranging a plurality of the X-ray detecting elements 24a, for example, 1,000 X-ray detecting elements in a channel direction CH (spread direction of X-ray beam 81). Herein, each of the detecting element rows are numbered 1, 2, 3, . . . , 64 from an end. This achieves a so-called 64-row multi-slice X-ray CT. However, the 64 detecting element rows herein are merely an example, and the present invention is not limited thereto. The X-ray detector 24 forms an X-ray detection surface 24s by the plurality of X-ray detecting elements 24a, the X-ray detection surface detecting the X-ray beam 81 that has passed through the subject 71. The X-ray detecting element 24a is configured as a so-called solid-state detector, for example, by combining a scintillator and a photodiode.

The central processing device 3 has a scan control unit 32, a pre-processing unit 34, and an image generating unit 35. The central processing device 3 is, for example, a processor such as a central processing unit (CPU) or the like. The central processing device 3 executes the functions of the scan control unit 32, pre-processing unit 34, and image generating unit 35 by reading and executing the program stored in the storing device 7. The program is an example of an embodiment of a control program according to the present invention.

The scan control unit 32 controls the X-ray control unit 22, the rotation control unit 26, the collimator control unit 27 and the imaging table 10 via the gantry control unit 29 so as to perform multi-energy imaging of the subject 71. Specifically, the scan control unit 32 controls the abovementioned units to rotate the X-ray tube 21 and the X-ray detector 24 around the subject 71 to acquire X-ray projection data.

In the present embodiment, target tube voltages are the first tube voltage V1 and the second tube voltage V2. For example, the first tube voltage V1 is 80 kV, and the second tube voltage V2 is 140 kV. In another embodiment, the first tube voltage V1 is 85 kV, and the second tube voltage V2 is 130 kV. In another embodiment, the first tube voltage V1 is 100 kV, and the second tube voltage V2 is 150 kV. Preferably, the first tube voltage V1 is 50 to 120 kV, and the second tube voltage V2 is 110 to 200 kV, which is higher than the first tube voltage V1. In the present embodiment, the difference between the first tube voltage V1 and the second tube voltage V2 is 20 to 100 kV, and preferably 40 to 70 kV. In another example, in addition to the first tube voltage V1 and the second tube voltage V2, a third tube voltage V3 is applied, and the third tube voltage V3 has a higher potential than the first tube voltage V1 and the second tube voltage V2. In this case, the effect of the fall time can be reduced by repeating low voltage, high voltage, and medium voltage. The number of types of tube voltages can be an arbitrary number equal to or greater than two, and in order to facilitate the reader's understanding, the following description will focus on an example in which dual-energy imaging is performed using only two types of tube voltages.

X-rays irradiated when the tube voltage of the X-ray tube 21 is the first tube voltage V1 are referred to as first X-rays, and X-rays irradiated when the tube voltage is the second tube voltage V2 are referred to as second X-rays. The first X-rays and the second X-rays have different tube voltages, and therefore, a first energy spectrum of the first X-ray and a second energy spectrum of the second X-ray are different spectra.

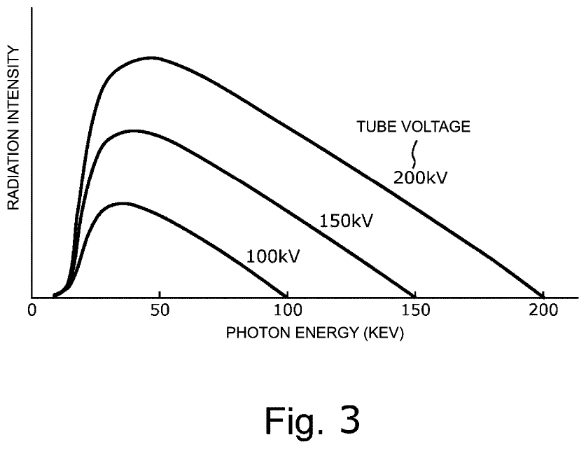

FIG. 3 depicts the energy spectrum of X-rays irradiated when representative tube voltages of 100 kV, 150 kV, and 200 kV are applied to the X-ray tube 21. As depicted in the drawing, whether the tube voltage is 100 kV, 150 kV, or 200 kV, the energy of the irradiated X-ray radiation is distributed as depicted in the drawing as energy lower than the applied kV.

In a preferred embodiment, the scan control unit 32 switches the X-ray tube voltage V for each view via the X-ray control unit 22, and the X-ray detector 24 accordingly acquires X-ray projection data for the number of views required for image reconstruction (X-ray projection data for a plurality of views equivalent to 180°+fan angle α or 360°). As a result, a first X-ray projection data p1 corresponding to the first tube voltage V1 and a second X-ray projection data p2 corresponding to the second tube voltage V2 are acquired. In another preferred embodiment, the X-ray control unit 22 switches the X-ray tube voltage V every two views. Sampling for generating the detection signal is performed a first number of times in each of the low voltage intervals and a second number of times in each of the high voltage intervals, and the first number of times and the second number of times may be the same or different.

The pre-processing unit 34 receives digital X-ray projection data from the DAS 25 and performs pre-processing on the X-ray projection data obtained by multi-energy imaging. Specifically, the first X-ray projection data p1 and the second X-ray projection data p2 are subjected to pre-processing such as offset correction, logarithmic conversion, sensitivity correction for correcting sensitivity non-uniformity between channels for raw data acquired by the data acquisition system 25, X-ray dose correction for correcting extreme reduction in signal intensity or signal loss due to metal parts and other strong X-ray absorbers, X-ray scattering correction, X-ray beam hardening correction, and the like. Furthermore, for pre-processing, the first X-ray projection data p1 and the second X-ray projection data p2 are subjected to a fan-parallel (fan-para) conversion for converting fan beam X-rays into parallel beam X-rays. For fan-parallel conversion, for example, the technique disclosed in Japanese Unexamined Patent Application S62-49831 can be used to prevent image degradation.

The image generating unit 35 generates a dual-energy image DE in which a specific substance is emphasized or suppressed, on the basis of the first X-ray projection data p1 and the second X-ray projection data p2 pre-processed by the pre-processing unit 34.

As a method for generating the dual-energy image DE, a method for performing weighted subtraction processing in an image data space and a method for performing weighted subtraction processing in a projection data space are conceivable, and either method may be adopted.

In the method of generating the dual-energy image DE by performing weighted subtraction processing in an image data space, a first image P1 is reconstructed on the basis of first X-ray projection data p1, a second image P2 is reconstructed on the basis of second X-ray projection data p2, and weighted subtraction processing is performed between the first image P1 and the second image P2 to generate the dual-energy image DE. On the other hand, in the method of generating the dual-energy image DE by performing weighted subtraction processing in a projection data space, weighted subtraction processing is performed on a view-by-view basis between the first X-ray projection data p1 and the second X-ray projection data p2, and the dual-energy image DE is reconstructed on the basis of the processed X-ray projection data obtained as a result.

Image reconstruction can be performed, for example, using a three-dimensional image reconstruction method based on the conventionally known Feldkamp method, other three-dimensional image reconstruction methods, two-dimensional image reconstruction methods, or the like, and is performed in accordance with the following procedure, for example. First, these X-ray projection data are subjected to a fast Fourier transform (FFT) for converting them into the frequency domain, and then multiplied by a reconstruction function Kernel (j) to perform an inverse Fourier transform. Furthermore, back projection processing is performed on the X-ray projection data multiplied by the reconstruction function Kernel (j) to obtain a tomographic image (xy plane) corresponding to the same slice when the subject 71 is sliced in the body axis direction (z-axis direction).

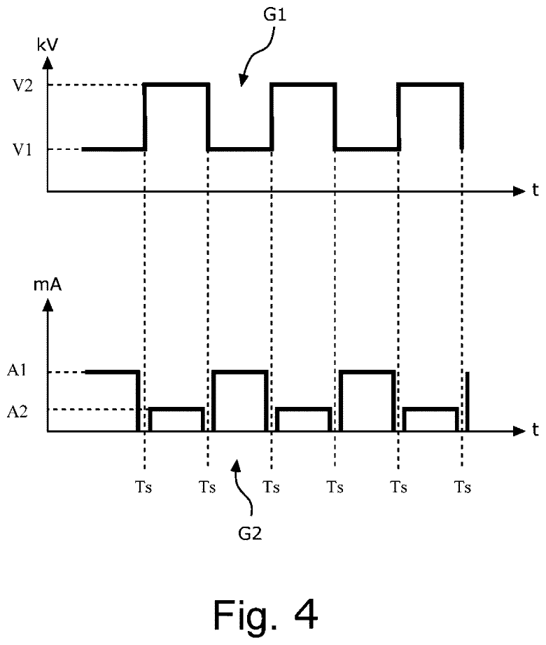

An operation of the X-ray CT device 100 according to the present embodiment will be described. The X-ray tube 21 and the X-ray detector 24 rotate around the body axis of the subject to acquire projection data. At this time, a switching control unit 321 switches the X-ray tube voltage of the X-ray tube 21 between the first tube voltage V1 and the second tube voltage V2, as depicted in graph G1 of FIG. 4. In graph G1, the horizontal axis represents time, and the vertical axis represents tube voltage. As a result, the X-ray tube 21 alternately irradiates the subject with the first X-rays and the second X-rays while rotating around the body axis of the subject. The voltage values of the first and second X-rays can be set by an operator using the input device 2.

The switching control unit 321 switches the X-ray tube current A of the X-ray tube 21 between the first tube current A1 and the second tube current, as depicted in graph G2 of FIG. 4. The first tube current A1 is the tube current when the X-ray tube 21 has the first tube voltage V1, and the second tube current A2 is the tube current when the X-ray tube 21 has the second tube voltage V2. For example, the first tube current A1 is 300 mA, and the second tube current A2 is 100 mA.

When the X-ray tube 21 is at the first tube voltage V1, the switching control unit 321 reduces the X-ray tube current A to be lower than the first tube current A1, and then switches the X-ray tube voltage from the first tube voltage V1 to the second tube voltage V2. After switching to the second tube voltage, the switching control unit 321 sets the tube current A to the second tube current A2. Furthermore, when the X-ray tube 21 is at the second tube voltage V2, the switching control unit 321 reduces the X-ray tube current A to be lower than for the second tube voltage V1, and then switches the X-ray tube voltage from the first tube voltage V1 to the second tube voltage V2. After switching to the first tube voltage, the switching control unit 321 sets the tube current A to the first tube current A1. In FIG. 4, the timing at which the first tube voltage V1 is switched to the second tube voltage V2 and the timing at which the second tube voltage V2 is switched to the first tube voltage V1 (switching timing) are indicated by the symbol Ts.

In this example, the tube current A is zero at the switching timing Ts. However, the tube current A at the switching timing Ts does not have to be zero. For example, the tube current A at the switching timing Ts may be about 1/100 of the first tube current A1 and the second tube current A2.

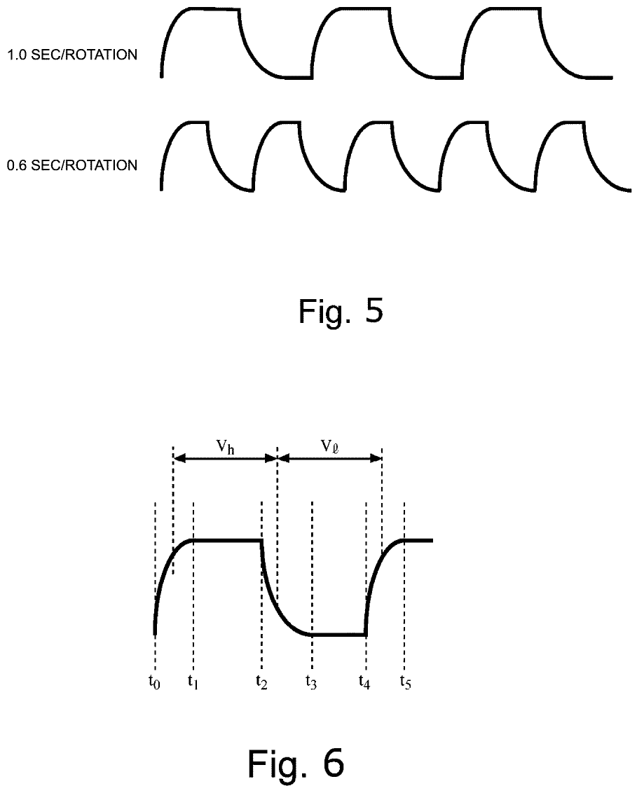

FIG. 5 is a diagram depicting the actual tube voltage applied to the X-ray tube 21. In FIGS. 5 and 6, the horizontal axis represents time, and the vertical axis represents voltage (kV). When switching the tube voltage between the first tube voltage V1 and the second tube voltage V2, it is ideal for the switching to be instantaneous as depicted in FIG. 4. However, due to limitations in the performance of transformers, rectifiers, and the like, in reality, voltage rising interval and falling interval occur as depicted in FIG. 5. Specifically, as depicted in the drawings, the tube voltage waveform includes a rising part from the low tube voltage to the high tube voltage, a falling part from the high tube voltage to the low tube voltage, a high steady-state interval between the rising part and the falling part, and a low steady-state interval between the falling part and the rising part.

In most CT devices, projection data of approximately 1000 views is acquired per rotation, regardless of the rotation speed. Therefore, when the rotation speed changes, the sampling rate and the cycle of switching the tube voltage also change. FIG. 5 depicts, as an example, tube voltage waveforms when the rotation speed is 1.0 sec/rotation and when the rotation speed is 0.6 sec/rotation. These tube voltage waveforms can be identified by applying an existing measuring device such as an oscilloscope or the like to the X-ray tube 21. In other embodiments, the number of views per rotation is set to approximately 2000, approximately 500, and the like, rather than approximately 1000. A fixed or variable number of views per rotation between 200 and 4000 are preferably acquired. The rotation speed and/or the number of views per rotation can be set by an operator using the input device 2.

As depicted in FIG. 5, when the rotation speed is 0.6 sec/rotation, there is no significant change in the rising and falling parts compared to when the rotation speed is 1.0 sec/rotation, but the high steady-state interval and low steady-state interval become shorter. The same applies when the number of views per rotation increases.

Furthermore, the tube voltage waveforms depicted in FIG. 5 are affected by the tube current. Specifically, when the current value of the power applied to the X-ray tube 21 is small, the voltage falling interval becomes relatively gentle, and when the current value is large, the voltage falling interval becomes relatively steep. In contrast, the voltage rising interval does not change significantly whether the current value is small or large. Such a change in the tube voltage waveform due to the tube current can also be identified by applying an existing measuring device such as an oscilloscope or the like to the X-ray tube 21. In a preferred embodiment, a tube current is applied to the X-ray tube 21 at a prescribed value within the range of 20 mA to 1000 mA. More preferably, a tube current of a prescribed value within the range of 50 mA to 500 mA is applied to the X-ray tube 21. Tube current switching can increase or decrease the current in units of 50 mA. In another embodiment, tube current switching can increase or decrease the current in units of 10 mA. The tube current values of the first and second X-rays can be set by an operator using the input device 2.

FIG. 6 depicts a portion of the tube voltage waveform. In FIG. 6, the voltage rising interval starts at t0 and ends at t1. The high steady-state interval starts at t1 and ends at t2. The voltage falling interval starts at t2 and ends at t3. The low steady-state interval starts at t3 and ends at t4. The repetition of these four intervals then continues in the same order.

When a detection signal of an X-ray beam based on such a voltage waveform is sampled, as depicted in FIG. 6, sampling can be performed with the interval from the start time between t0 and t1 to the end time between t2 and t3 as a high X-ray energy interval (high tube voltage interval Vh), and the interval from the start time between t2 and t3, which starts simultaneously with the end of the high tube voltage period Vh, to the end time between t4 and t5 as a low X-ray energy interval (low tube voltage interval Vl). In a preferred embodiment, the duration of the high tube voltage interval Vh and the low tube voltage interval Vl are the same. In another embodiment, the duration of the high tube voltage interval Vh is 0 to 20% longer than the duration of the low tube voltage interval Vl. In another embodiment, the duration of the low tube voltage interval Vl is 0 to 20% longer than the duration of the high tube voltage interval Vh.

The timing at which the high tube voltage interval Vh depicted in FIG. 6 starts (corresponding to the end of the low tube voltage interval Vl) and the timing at which the high tube voltage interval Vh ends (corresponding to the start of the low tube voltage interval Vl) can be changed in various manners. However, if the high tube voltage interval Vh starts at time to and ends at time t2, the high tube voltage interval Vh includes a voltage portion in which the value is close to the low V1, while the low tube voltage interval Vl includes a voltage portion in which the value is close to the high V2, and thus is not preferable. In both the voltage rising interval and the voltage falling interval, it is desirable to include the high voltage portion in the high tube voltage interval Vh, and the low voltage portion in the low tube voltage interval Vl. In the example of FIG. 6, one sampling is performed in the high tube voltage interval Vh and one sampling is performed in the low tube voltage interval Vl, but a similar problem occurs when two or more samplings are performed in the high tube voltage interval Vh and two or more samplings are performed in the low tube voltage interval Vl. Furthermore, starting the low tube voltage interval Vl simultaneously with the end of the high tube voltage interval Vh has the advantages of minimizing the radiation dose to the imaging subject, and the like, but problems with the voltage rising interval and voltage falling interval also occur when a short time interval is provided between the end of the high tube voltage interval Vh and the start of the low tube voltage interval Vl and/or between the end of the low tube voltage interval Vl and the start of the high tube voltage interval Vh.

In order to include the high voltage portion in the high tube voltage interval Vh and the low voltage portion in the low tube voltage interval Vl, both in the voltage rising interval and in the voltage falling interval, the start time of the high tube voltage interval Vh can be set to a prescribed delay time after the time to at which the application of the second tube voltage V2 starts. In another embodiment, a time point at which the tube voltage exceeds a prescribed threshold value can be set as the start time of the high tube voltage interval Vh. The end time of the high tube voltage interval Vh can be set to the time point when half the repetition cycle (or a prescribed time interval such as 55% of the repetition cycle or the like) has elapsed from the start time of the high tube voltage interval Vh. In another embodiment, a time point at which the tube voltage falls below a prescribed threshold value can be set as the end time of the high tube voltage interval Vh. The timing of sampling performed by the DAS 25 can be set on the basis of the results of such automatic analysis of such voltage waveform.

As described above, when the rotation speed increases and/or the number of views per rotation increases, the steady-state interval of the tube voltage waveform becomes shorter. Moreover, when the current value of the power applied to the X-ray tube 21 is small, the voltage falling interval becomes relatively gradual, and when the current value is large, the voltage falling interval becomes relatively steep. Therefore, regardless of how the start time and end time of the high tube voltage interval Vh are set, the energy of the first X-ray based on the tube voltage in the low tube voltage interval Vl and the energy of the second X-ray based on the tube voltage in the high tube voltage interval Vh vary over time. Therefore, the average energy or energy average value of the first X-rays and the energy average value of the second X-rays also vary depending on the rotation speed, the number of views per rotation, and the tube current. Note that the average value referred to in the present specification does not necessarily mean an average of pure energy values, but may be calculated by adding corrections that take into account X-ray absorption properties or may be based on a representative value.

For example, when the first tube voltage V1 is 100 kV and the second tube voltage V2 is 150 kV, X-rays having the energy distribution indicated for a tube voltage of 100 kV in FIG. 3 are irradiated at time t0 in FIG. 6, and the energy distribution rapidly changes to the energy distribution indicated for a tube voltage of 150 kV in FIG. 3 toward time t1. From time t1 to time t2, irradiation of X-rays having the energy distribution indicated at a tube voltage of 150 kV in FIG. 3 is maintained, and from time t2 to time t3, the energy distribution changes from the energy distribution indicated at a tube voltage of 150 kV in FIG. 3 to the energy distribution indicated at a tube voltage of 100 kV in FIG. 3, initially abruptly and then gradually toward the end. Furthermore, from time t3 to time t4, irradiation of X-rays having the energy distribution indicated in FIG. 3 at a tube voltage of 100 kV is maintained.

Furthermore, if the rotation speed is faster or the number of views per rotation is greater, the time interval between times t1 and t2 and the time interval between times t3 and t4 become shorter, and if the rotation speed is slower or the number of views per rotation is smaller, the time interval between times t1 and t2 and the time interval between times t3 and t4 become longer. Furthermore, whether the magnitude of the tube current is large or small, there is no significant change in the time interval between time t0 and time t1 and the time interval between time t1 and time t2. In other words, regardless of the magnitude of the tube current, the tube voltage waveform forms a relatively steep rising curve between time t0 and time t1. When the magnitude of the tube current is large, the time interval between time t2 and time t3 becomes short, and the time interval between time t3 and time t4 becomes long. On the other hand, when the magnitude of the tube current is small, the time interval between time t2 and time t3 becomes long, and the time interval between time t3 and time t4 becomes short. In other words, when the magnitude of the tube current is small, the tube voltage waveform forms a gentle descending curve between time t2 and time t3.

The inventors of the present invention have discovered that the aforementioned changes in the tube voltage waveform have regularity and can be modeled. Specifically, the tube voltage waveform is specified as follows.

Time t1: A point in time determined by a polynomial that is a function of the potential difference between the second tube voltage V2 and the first tube voltage V1.

Times t2, t4: Points in time identified by the rotation speed and/or the number of views per rotation (e.g., if the rotation speed is 1.0 sec/rotation and 1000 views are acquired per rotation, t2 is 1 ms after t0 and t4 is 2 ms after t0, and if the rotation speed is 1.0 sec/rotation and 500 views are acquired per rotation, t2 is 2 ms after t0 and t4 is 4 ms after t0).

Time t3: A point in time determined by a polynomial that is a function of the potential difference between the second tube voltage V2 and the first tube voltage V1 and the magnitude of the tube current.

Between time t0 and time t1: A curve specified by a polynomial that is a function of the potential difference between the second tube voltage V2 and the first tube voltage V1. When the potential difference between the second tube voltage V2 and the first tube voltage V1 is large, the time interval between time t0 and time t1 becomes long. When the potential difference between the second tube voltage V2 and the first tube voltage V1 is small, the time interval between time t0 and time t1 becomes short.

Between time t1 and time t2: A straight line identified by the second tube voltage V2.

Between time t2 and time t3: A curve specified by a polynomial that is a function of the potential difference between the second tube voltage V2 and the first tube voltage V1 and the magnitude of the tube current. When the potential difference between the second tube voltage V2 and the first tube voltage V1 is large, the time interval between time t2 and time t3 becomes long. When the potential difference between the second tube voltage V2 and the first tube voltage V1 is small, the time interval between time t2 and time t3 becomes short. If the tube current is large, the time interval between time t2 and time t3 becomes short. If the tube current is small, the time interval between time t2 and time t3 becomes long.

Between time t3 and time t4: A straight line identified by the first tube voltage V1.

In this embodiment, the curved section of the tube voltage waveform is modeled using a polynomial, but in other embodiments, modeling is possible using a mathematical model for approximation by a combination of a linear function, a quadratic function, and a cubic function.

The tube voltage waveform can also be modeled as an artificial intelligence (AI) model constructed using deep learning. In deep learning, a trained AI model can be constructed by repeatedly learning combinations of the first tube voltage V1, the second tube voltage V2, the tube current, the rotation speed, and the number of views/rotations, as well as the actual tube voltage waveforms measured under such combinations, as training data.

On the other hand, beam hardening correction has been conventionally performed on image data acquired by multi-energy imaging. The beam hardening correction is based on the phenomenon called a “beam hardening effect”, whereby the average energy of the X-rays emitted by a penetrated object shifts to a higher energy value in the case of polychromatic X-rays due to the spectral correlation of the light attenuation performance of a real object. In a reconstructed image of the object, the linear and spectrally related light attenuation can be observed via a shift relative to a grayscale value in a theoretical case. In particular, a shift in a grayscale value in the reconstructed image caused by high nuclear charge numbers and high density materials (such as a bone or the like) or beam hardening virtual images can cause the reconstructed image to interfere with correct interpretation of the image.

In order to reduce or remove this beam hardening effect, beam hardening correction is applied to image data acquired by multi-energy imaging, and in this case, a beam hardening correction coefficient is used. The beam hardening correction coefficient is obtained by performing calibration in which a phantom, such as a water phantom or the like, is actually scanned by multi-energy imaging. During calibration, the CT value of water is set to 0, and the CT value of air is set to −1000. However, as described above, the average energy of the X-rays varies depending on the rotation speed, the number of views per rotation, the tube current, and the potential difference between the second tube voltage V2 and the first tube voltage V1. Therefore, conventionally, in order to correctly set the beam hardening correction coefficient, calibration was performed using a combination of the rotation speed, the number of views per rotation, the tube current, and the potential difference between the second tube voltage V2 and the first tube voltage V1 to be used.

However, by modeling the tube voltage waveform as described above, it is possible to identify a tube voltage waveform corresponding to a specific combination of the rotation speed, the number of views per rotation, the tube current, and the potential difference between the second tube voltage V2 and the first tube voltage V1. When the tube voltage waveform can be identified, the average energy of the X-ray beam based on the tube voltage waveform can be identified, and the beam hardening correction coefficient to be applied can be obtained.

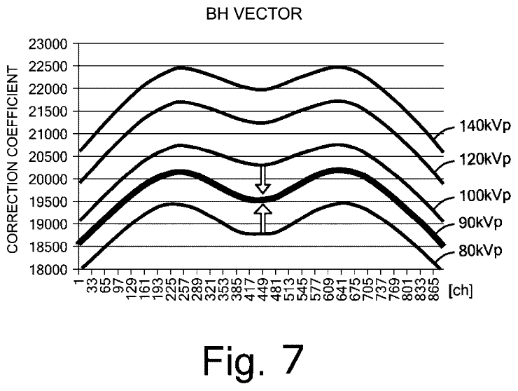

FIG. 7 is a diagram describing a technique for obtaining a beam hardening correction coefficient to be applied. When the beam hardening correction coefficient is embodied by a beam hardening vector (BH vector) corresponding to a detector channel, a BH vector for an X-ray beam having an energy corresponding to a major tube voltage, such as 80 kVp, 100 kVp, 120 kVp, 140 kVp, or the like, can be obtained using an existing technique, such as calibration imaging using a phantom, or the like.

Furthermore, if the average energy of the X-ray beam obtained by modeling the tube voltage waveform is, for example, 90 kVp, a BH vector of 90 kVp can be obtained by using the average of the BH vector of 80 kVp and the BH vector of 100 kVp, as depicted in FIG. 7. Furthermore, in another example, if the desired BH vector is between 120 kVp and 140 kVp (X kVp), the BH vector for X kVp can be obtained by using a weighted average of the BH vectors for 120 kVp and 140 kVp. In such a case, the value of the BH vector of X kVp can be as follows for each channel:

BH vector value of X kVp=((140−X)×120 kVp BH vector value+(X−120)×140 kVp BH vector value)/20 (120<X<140).

Furthermore, it is known to a person of ordinary skill in the art that the energies of scattered X-rays and recoil electrons generated by Compton scattering change depending on the energy of the incident X-rays, and that the distribution of the scattered X-rays also changes depending on the energy of the incident X-rays. In order to remove or reduce the influence of such scattered X-rays, scattering correction has conventionally been performed. The scattering correction is particularly important in dental CT devices and CT devices for baggage inspection, where a scanned object is likely to contain metal. Coefficients used for the scattering correction can also be calculated on the basis of the average value of the X-ray beam calculated from the tube voltage waveform model. Furthermore, the average value of the X-ray beam can be used to calculate an absorption value u of the basis material pairs used in material decomposition (basis material decomposition) calculations.

Furthermore, the rate at which X-rays are absorbed varies depending on the type and concentration of elements that make up the imaging subject, and the amount of X-rays received by the detector decreases when substances with high atomic numbers are present at high density. Furthermore, it is known to a person of ordinary skill in the art that the degree of absorption changes depending on the energy of the incident X-rays. In order to remove or reduce the influence of such absorbed X-rays, absorption correction has conventionally been performed. The absorption correction is particularly important in dental CT devices and CT devices for baggage inspection, where a scanned object is likely to contain metal. Coefficients used for the absorption correction can also be calculated on the basis of the average value of the X-ray beam calculated from the tube voltage waveform model.

Any of the beam hardening correction coefficients, scattering correction coefficients, and absorption correction coefficients can be generated for each of the plurality of detector elements 24a or for a group of the plurality of detector elements 24a. The image generating unit 35 can also reconstruct an image using the difference between the beam hardening correction coefficient calculated by the pre-processing unit 34 from the tube voltage waveform model and the beam hardening correction coefficient calculated by imaging a phantom.

Thus, by setting the parameter used to create a radiological image in accordance with the low-energy average value and the high-energy average value calculated from the tube voltage waveform model, the imaging and calculation time for calibration can be shortened. This reduces CT device downtime and service costs.

In a preferred embodiment of the present invention, a function for updating the tube voltage waveform model is provided. Due to repeated use of the CT device or deterioration over time, the shape of the tube voltage waveform may change due to factors such as the waveform of the voltage output by a power supply device, deterioration of the target of the X-ray tube 21, or the like. Moreover, even if the tube voltage waveform does not change, the energy distribution of the X-ray beam irradiated may change on the basis thereof. In such a case, the tube voltage waveform model generated at the time of shipment, at the start of use, or the like may no longer be compatible with a current CT device. In such a case, the tube voltage waveform model is corrected on the basis of the projection data measured during calibration.

Note that the invention is not limited to the present embodiment, and various modifications are possible without departing from the gist of the invention.

Furthermore, a program for causing a computer to function as each means for controlling and processing the X-ray CT device is also an example of an embodiment of the invention.

1. A system, comprising:
a processor for processing a detection signal of radiation transmitted through an imaging subject to generate a radiological image, wherein
the radiation is irradiated toward the imaging subject while a radiation tube rotates around the imaging subject;
the radiation includes low-energy radiation generated by applying a low tube voltage to the radiation tube and high-energy radiation generated by applying a high tube voltage to the radiation tube;
application of the low tube voltage and the high tube voltage to the radiation tube are alternately switched during rotation of the radiation tube, to form a tube voltage waveform having a rising part from the low tube voltage to the high tube voltage, a falling part from the high tube voltage to the low tube voltage, a high steady-state interval between the rising part and the falling part, and a low steady-state interval between the falling part and the rising part; and
the processer to execute the following:
receives input for rotation speed and/or number of views per rotation,
identifies a tube voltage waveform corresponding to the rotation speed and/or the number of views per rotation using a waveform identification model,
calculates a low-energy average value of the radiation corresponding to a low voltage interval including the low steady-state interval of the tube voltage waveform and a part of the falling part of the tube voltage waveform, and a high-energy average value of the radiation corresponding to a high voltage interval including the high steady-state interval of the tube voltage waveform and another part of the falling part of the tube voltage waveform, and
sets a parameter used for creating the radiological image in accordance with the low-energy average value and the high-energy average value.

2. The system according to claim 1, further including:
a table on which the imaging subject is placed;
a gantry for rotatably supporting the radiation tube and a detector that detects the radiation transmitted through the imaging subject;
a storing medium for storing the waveform identification model;
a user interface for inputting the rotation speed and/or the number of views per rotation; and
an image reconstructing device including the processor.

3. The system according to claim 1, wherein the parameter includes a beam hardening correction coefficient.

4. The system according to claim 1, wherein the parameter includes an X-ray absorption coefficient and/or an X-ray scattering coefficient.

5. The system according to claim 1, wherein the low voltage interval includes a part of a rising part of the tube voltage waveform,
the high voltage interval includes another part of a rising part of the tube voltage waveform, and
the waveform identification model makes the voltage falling part relatively gradual when a current value of the power applied to the radiation tube is low and makes the voltage falling part relatively steep when the current value is high, while not significantly changing the voltage rising part when the current value is low or high.

6. The system according to claim 1, wherein the tube voltage waveform is acquired by measuring power applied to the radiation tube, and
in the waveform identification model, a curve of a voltage falling part of a measured waveform is expressed by an approximation of a polynomial.

7. The system according to claim 6, wherein the processor
divides the tube voltage waveform into the low voltage interval and the high voltage interval by a prescribed threshold value,
sampling for generating the detection signal is performed a first number of times in each of the low voltage intervals and a second number of times in each of the high voltage intervals, and
the first number of times is the same as the second number of times.

8. The system according to claim 7, wherein the detector includes a plurality of detector elements extending in a circumferential direction of the gantry,
the radiation tube irradiates the detector with a fan beam or a cone beam diverging in the circumferential direction as the radiation,
the detector detects the radiation transmitted through a phantom to generate a phantom detection signal,
a first phantom-based beam hardening correction coefficient corresponding to the low-energy radiation is generated on the basis of the phantom detection signal,
the first phantom-based beam hardening correction coefficient is generated for each of the plurality of detector elements,
the parameter includes a first model-based beam hardening correction coefficient corresponding to the low voltage interval, and
the radiological image is reconstructed using a first difference between the first model-based beam hardening correction coefficient and the first phantom-based beam hardening correction coefficient.

9. The system according to claim 8, wherein a second phantom-based beam hardening correction coefficient corresponding to the high-energy radiation is generated on the basis of the phantom detection signal,
the second phantom-based beam hardening correction coefficient is generated for each of the plurality of detector elements,
the parameter includes a second model-based beam hardening correction coefficient corresponding to the low voltage interval, and
the radiological image is reconstructed using a second difference between the second model-based beam hardening correction coefficient and the second phantom-based beam hardening correction coefficient.

10. A program for processing a detection signal of radiation transmitted through an imaging subject in order to generate a radiological image, wherein
the radiation is irradiated toward the imaging subject while a radiation tube rotates around the imaging subject,
the radiation includes low-energy radiation generated by applying a low tube voltage to the radiation tube and high-energy radiation generated by applying a high tube voltage to the radiation tube;
application of the low tube voltage and the high tube voltage to the radiation tube are alternately switched during rotation of the radiation tube, to form a tube voltage waveform having a rising part from the low tube voltage to the high tube voltage, a falling part from the high tube voltage to the low tube voltage, a high steady-state interval between the rising part and the falling part, and a low steady-state interval between the falling part and the rising part; and
the program causes a processor to execute the following:
receiving input for rotation speed and/or number of views per rotation;
identifying a tube voltage waveform corresponding to the rotation speed and/or the number of views per rotation using a waveform identification model;
calculating a low-energy average value of the radiation corresponding to a low voltage interval including the low steady-state interval of the tube voltage waveform and a part of the falling part of the tube voltage waveform, and a high-energy average value of the radiation corresponding to a high voltage interval including the high steady-state interval of the tube voltage waveform and another part of the falling part of the tube voltage waveform; and
setting a parameter used for creating the radiological image in accordance with the low-energy average value and the high-energy average value.

11. The program according to claim 10, wherein the imaging subject is placed on a table,
the radiation transmitted through the imaging subject is detected by a detector,
the detector and the radiation tube are rotatably supported by a gantry,
the waveform identification model is pre-stored in a storing medium, and
the rotation speed and/or the number of views per rotation are input via a user interface.

12. The program according to claim 10, wherein the parameter includes a beam hardening correction coefficient.

13. The program according to claim 10, wherein the parameter includes an X-ray absorption coefficient and/or an X-ray scattering coefficient.

14. The program according to claim 10, wherein the low voltage interval includes a part of a rising part of the tube voltage waveform,
the high voltage interval includes another part of a rising part of the tube voltage waveform, and
the waveform identification model makes the voltage falling part relatively gradual when a current value of the power applied to the radiation tube is low and makes the voltage falling part relatively steep when the current value is high, while not significantly changing the voltage rising part when the current value is low or high.

15. The program according to claim 11, wherein the tube voltage waveform is acquired by measuring power applied to the radiation tube, and
in the waveform identification model, a curve of a voltage falling part of a measured waveform is expressed by an approximation of a polynomial.

16. The program according to claim 15, wherein the program causes the processor to execute dividing the tube voltage waveform into the low voltage interval and the high voltage interval using a prescribed threshold value,
sampling for generating the detection signal is performed a first number of times in each of the low voltage intervals and a second number of times in each of the high voltage intervals, and
the first number of times is the same as the second number of times.

17. The program according to claim 16, wherein the detector includes a plurality of detector elements extending in a circumferential direction of the gantry,
the radiation tube irradiates the detector with a fan beam or a cone beam diverging in the circumferential direction as the radiation,
the detector detects the radiation transmitted through a phantom to generate a phantom detection signal,
a first phantom-based beam hardening correction coefficient corresponding to the low-energy radiation is generated on the basis of the phantom detection signal,
the first phantom-based beam hardening correction coefficient is generated for each of the plurality of detector elements,
the parameter includes a first model-based beam hardening correction coefficient corresponding to the low voltage interval, and
the radiological image is reconstructed using a first difference between the first model-based beam hardening correction coefficient and the first phantom-based beam hardening correction coefficient.

18. The program according to claim 17, wherein a second phantom-based beam hardening correction coefficient corresponding to the high-energy radiation is generated on the basis of the phantom detection signal,
the second phantom-based beam hardening correction coefficient is generated for each of the plurality of detector elements,
the parameter includes a second model-based beam hardening correction coefficient corresponding to the low voltage interval, and
the radiological image is reconstructed using a second difference between the second model-based beam hardening correction coefficient and the second phantom-based beam hardening correction coefficient.
